(19) United States
(12) Patent Application Publication
(10) Pub. No.: US 2023/0107649 A1
(43) Pub. Date: Apr. 6, 2023

(54) AUTOMATIC EYE TEST DEVICE AND AUTOMATIC EYE TEST METHOD

(71) Applicant: Crystalvue Medical Corporation, Taoyuan City (TW)

(72) Inventors: William WANG, Taoyuan City (TW); Chung-Ping CHUANG, Taoyuan City (TW); Chun Nan LIN, Bade City (TW); Chung-Cheng CHOU, Luzhu Township (TW)

(21) Appl. No.: 17/874,348

(22) Filed: Jul. 27, 2022

(30) Foreign Application Priority Data
Oct. 1, 2021 (TW) 110136783

(51) Int. Cl. A61B 3/10; A61B 3/00

(52) U.S. Cl. CPC A61B 3/102; A61B 3/0016; A61B 3/12

(57) ABSTRACT

An automatic eye test device and an automatic eye test method are disclosed. The automatic eye test device includes an automatic positioning module, an automatic test module and an automatic analysis module. The automatic positioning module is configured to automatically complete positioning of an eye of a person to be tested. The automatic test module is coupled to the positioning module and configured to automatically test the eye of the person to be tested to obtain a test result. The automatic analysis module is coupled to the automatic test module and configured to automatically analyze the test result to provide a personalized test suggestion for the person to be tested.

### CROSS-REFERENCE TO RELATED APPLICATIONS

This application claims the benefit of Taiwan patent application No. 110136783 filed on Oct. 1, 2021, and the entire content of which is hereby incorporated by reference herein in its entirety.

### BACKGROUND OF THE INVENTION

### Field of the Invention

The invention relates to eye test; in particular, to an automatic eye test device and an automatic eye test method.

### Description of the Prior Art

Conventionally, when ordinary people want to perform different eye tests items, they often need professional tester to operate multiple test instruments with different test functions to test the eyes of the person to be tested. The person to be tested cannot complete the entire eye test procedure alone. The procedure not only makes the person to be tested and the tester feel very time-consuming and labor-intensive, but also causes great limitations and inconveniences to the popularity and convenience of eye test for the general public. Therefore, it is urgent to solve the above-mentioned problems.

### SUMMARY OF THE INVENTION

Therefore, the invention provides an automatic eye test device and an automatic eye test method to solve the above-mentioned problems of the prior arts.

A preferred embodiment of the invention is an automatic eye test device. In this embodiment, the automatic eye test device includes an automatic positioning module, an automatic test module and an automatic analysis module. The automatic positioning module is configured to automatically complete a positioning of an eye of a person to be tested. The automatic test module is coupled to the positioning module and configured to automatically test the eye of the person to be tested to obtain a test result. The automatic analysis module is coupled to the automatic test module and configured to automatically analyze the test result to provide a personalized test suggestion for the person to be tested.

In an embodiment, the automatic positioning module senses at least two feature points of a face of the person to be tested and compares the at least two feature points with a plurality of feature point and eye corresponding position information stored in a database to complete the positioning of the eye of the person to be tested.

In an embodiment, the automatic positioning module determines whether the person to be tested has used the automatic eye test device according to an identification information provided by the person to be tested; if a determination result of the automatic positioning module is yes, the automatic positioning module finds out an eye positioning information corresponding to the person to be tested from a database to complete the positioning of the eye of the person to be tested; if the determination result is no, the automatic positioning module compares at least one identification item in the identification information with a plurality of identification item and eye corresponding position information stored in the database to complete the positioning of the eye of the person to be tested.

In an embodiment, the at least two feature points are selected from a forehead, a chin, a left ear, a right ear, a left cheek, a right cheek, a left cheekbone, a right cheekbone, a nose tip, a nose bridge, a left mouth corner, a right mouth corner, an upper lip and a lower lip.

In an embodiment, the personalized test suggestion includes a suggestion for a testing appointment date and/or a suggestion for testing items.

In an embodiment, the automatic test module performs vision test, optometry, fundus photography, retinal optical coherence tomography (OCT), intraocular pressure test, corneal thickness test and/or corneal curvature test on the eye of the person to be tested.

In an embodiment, when the test result is determined to be unreliable, the automatic test module automatically retests the eye of the person to be tested.

In an embodiment, the automatic positioning module is disposed on a six-degree-of-freedom (6-DOF) motion platform to operate.

Another preferred embodiment of the invention is an automatic eye test method. In this embodiment, the automatic eye test method includes steps of: (a) automatically completing a positioning of an eye of a person to be tested; (b) automatically test the eye of the person to be tested to obtain a test result; and (c) automatically analyze the test result to provide a personalized test suggestion for the person to be tested.

In an embodiment, the step (a) includes: sensing at least two feature points of a face of the person to be tested and comparing the at least two feature points with a plurality of feature point and eye corresponding position information stored in a database to complete the positioning of the eye of the person to be tested.

In an embodiment, the step (a) includes: determining whether the person to be tested has used the automatic eye test device according to an identification information provided by the person to be tested; if a determination result of the step (a) is yes, finding out an eye positioning information corresponding to the person to be tested from a database to complete the positioning of the eye of the person to be tested; if the determination result is no, comparing at least one identification item in the identification information with a plurality of identification item and eye corresponding position information stored in the database to complete the positioning of the eye of the person to be tested.

In an embodiment, the at least two feature points are selected from a forehead, a chin, a left ear, a right ear, a left cheek, a right cheek, a left cheekbone, a right cheekbone, a nose tip, a nose bridge, a left mouth corner, a right mouth corner, an upper lip and a lower lip.

In an embodiment, the personalized test suggestion includes a suggestion for a testing appointment date and/or a suggestion for testing items.

In an embodiment, the step (b) includes: performing vision test, optometry, fundus photography, retinal optical coherence tomography (OCT), intraocular pressure test, corneal thickness test and/or corneal curvature test on the eye of the person to be tested.

Compared to the prior art, the automatic eye test device and method of the invention can automatically identify the identity of the person to be tested to obtain his/her previous eye positioning information to automatically complete the positioning of the eye of the person to be tested and automatically perform different tests on the eye of the person to be tested to obtain the test result, and they can also automatically analyze the personalized test suggestion (such as a suggestion for a testing appointment date and/or a suggestion for testing items) for the person to be tested based on the test result, so the person to be tested can use a single automatic eye test device to complete the entire eye test procedure by himself/herself. There is no need for professional testers to test the person to be tested through multiple eye test instruments, so it can greatly reduce the cost of manpower and equipment and effectively improve the popularity and convenience of eye test.

The advantage and spirit of the invention may be understood by the following detailed descriptions together with the appended drawings.

### BRIEF DESCRIPTION OF THE APPENDED DRAWINGS

FIG. 1 illustrates a schematic diagram of an automatic eye test device in an embodiment of the invention.

FIG. 2 illustrates a schematic diagram showing that the automatic positioning module completes the positioning of the eyes of the person to be tested according to the at least three feature points of the face of the person to be tested.

FIG. 3 illustrates a flowchart of an automatic eye test method in another embodiment of the invention.

### DETAILED DESCRIPTION OF THE INVENTION

Exemplary embodiments of the invention are referenced in detail now, and examples of the exemplary embodiments are illustrated in the drawings. Further, the same or similar reference numerals of the components/components in the drawings and the detailed description of the invention are used on behalf of the same or similar parts.

A specific embodiment of the invention is an automatic eye test device. In this embodiment, the automatic eye test device is used to perform a variety of automatic test functions on eyes of a person to be tested. It can be disposed in different positions in the living environment in a similar manner to a cash machine, so that the general public can find time to complete the entire eye test procedure alone to effectively enhance the popularity and convenience of eye test for the general public.

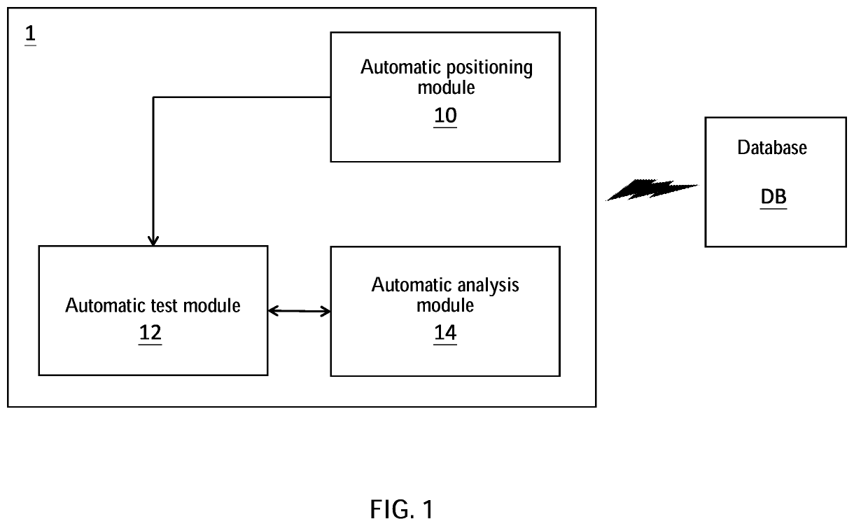

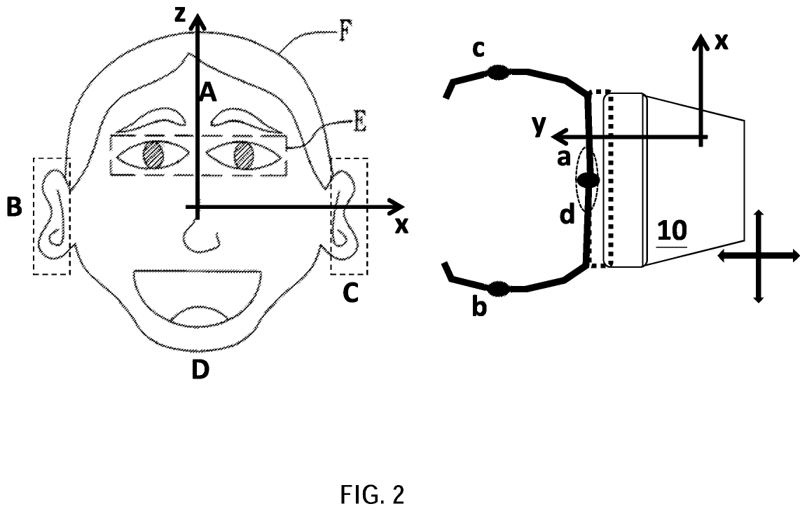

Please refer to FIG. 1. FIG. 1 illustrates a schematic diagram of the automatic eye test device in this embodiment. As shown in FIG. 1, the automatic eye test device 1 includes an automatic positioning module 10, an automatic test module 12 and an automatic analysis module 14. The automatic eye test device 1 can be coupled to a database DB. The automatic positioning module 10 is used to automatically complete the positioning of the eyes of the person to be tested (for example, as shown in FIG. 2, the automatic positioning module 10 can sense two or more feature points of the face of the person to be tested and the positioning of the eyes of the person to be tested can be completed according to these feature points, but not limited to this). The automatic test module 12 is coupled to the automatic positioning module 10 to automatically test the eyes of the person to be tested to obtain the test results. The automatic analysis module 14 is coupled to the automatic test module 12 to automatically analyze the test results to provide personalized test suggestions for the person to be tested.

In practical applications, the automatic eye test device 1 can be coupled to the database DB through any network or wireless transmission protocol, or the database DB can be disposed in a memory of the automatic eye test device 1.

In an embodiment, the automatic eye test device 1 can directly use the big data stored in the database DB to identify the identity of the person to be tested to complete the positioning of the eyes of the person to be tested. For example, the automatic positioning module 10 can automatically sense at least three feature points of the face of the person to be tested and then compare them with a plurality of feature point and eye corresponding position information stored in the database DB to find out the corresponding eye position information, and then the positioning of the eyes of the person to be tested can be completed according to the corresponding eye position information, but not limited to this.

In another embodiment, the automatic eye test device 1 can also perform the identity comparison of the person to be tested first, and then perform the positioning of the eyes of the person to be tested. For example, the automatic positioning module 10 can first determine whether the person to be tested has used the automatic eye test device 1 before according to the identification information (such as name, date of birth, fingerprint, voiceprint, retina, identity card, etc.) provided by the person to be tested.

If the determination result of the automatic positioning module 10 is yes, it means that the person to be tested has used the automatic eye test device 1 before. Therefore, the database DB should store the eye position information of the person to be tested, and the automatic positioning module 10 can automatically find out the eye positioning information corresponding to the person to be tested from the database DB, and then the positioning of the eyes of the person to be tested can be completed according to the corresponding eye position information, but not limited to this.

If the determination result of the automatic positioning module 10 is no, it means that the person to be tested has not used the automatic eye test device 1 before. Therefore, the eye location information of the person to be tested will not be stored in the database DB. Then, the automatic positioning module 10 can compare at least one identity item (such as gender, age, race, etc.) in the identity information with a plurality of identity item and eye position corresponding information stored in the database DB to obtain the eye position information corresponding to the person to be tested from the database DB, and then the positioning of the eyes of the person to be tested can be completed according to the corresponding eye position information, but not limited to this.

In practical applications, the automatic positioning module 10 automatically senses at least three feature points of the face of the person to be tested, which can be selected from a forehead, a chin, a left ear, a right ear, a left cheek (a left cheekbone), a right cheek (a right cheekbone), a nose tip, a nose bridge, a left mouth corner, a right mouth corner, an upper lip and a lower lip, but not limited to this.

For example, as shown in FIG. 2, if A to E in the two-dimensional space of the X direction and the Z direction represent the forehead, right ear, left ear, chin, and eyes of the face F of the person to be tested respectively, and A to D correspond to the positioning points a to d in the two-dimensional space of the X direction and the Y direction respectively, the automatic positioning module 10 can use three feature points of A-a, B-b and C-c to position the eye E, or use three feature points of D-d, B-b and C-c to position the eye E, or use three feature points of A-a, B-b and D-d to position the eye E, or use three feature points of A-a, C-c and D-d to position the eye E, or use four feature points of A-a, B-b, C-c, and D-d to position the eye E, but not limited to this.

It should be noted that, in order to complete the positioning of the eyes of the person to be tested, the automatic positioning module 10 needs to be able to move in different dimensions. For example, the automatic positioning module 10 can be disposed on a six-degree-of-freedom (6-DOF) motion platform to operate, but not limited to this.

After the automatic positioning module 10 completes the positioning of the eyes of the person to be tested, the automatic test module 12 will automatically test the eyes of the person to be tested to obtain the test result. In practical applications, the automatic test module 12 can be a multifunctional ophthalmological test device, which can perform different tests on the eyes of the person to be tested according to actual needs, such as vision test, optometry, fundus photography, retinal optical coherence tomography (OCT), intraocular pressure test, corneal thickness test and/or corneal curvature test, but not limited to this.

It should be noted that when the test result provided by the automatic test module 12 is determined to be unreliable, the automatic test module 12 will automatically retest the eyes of the person to be tested to obtain a retest result.

When the automatic test module 12 provides the test result, the automatic analysis module 14 will automatically analyze the test result to provide personalized test suggestions for the person to be tested, such as a suggestion for a testing appointment date and/or a suggestion for testing items, but not limited to this.

Another specific embodiment of the invention is an automatic eye test method. In this embodiment, the automatic eye test method can be applied to an automatic eye test device to perform a variety of automatic test functions on eyes of a person to be tested. It can be disposed in different positions in the living environment in a similar manner to a cash machine for the general public to find time to complete the entire eye test procedure alone, it can effectively enhance the popularity and convenience of eye test for the general public.

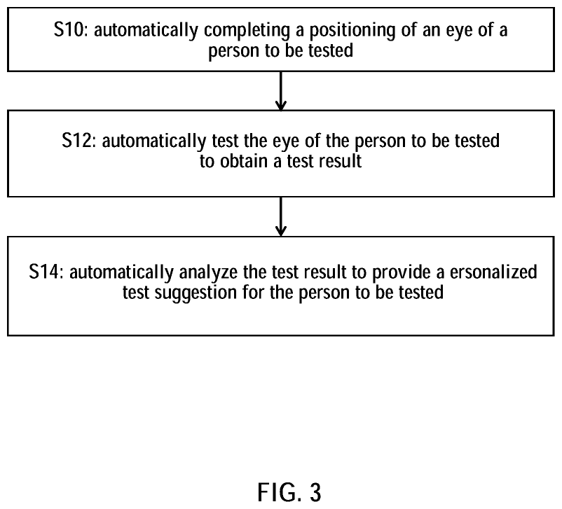

Please refer to FIG. 3. FIG. 3 illustrates a flowchart of the automatic eye test method of this embodiment. As shown in FIG. 3, the automatic eye test method can include the following steps:

Step S10: automatically completing a positioning of an eye of a person to be tested;

Step S12: automatically test the eye of the person to be tested to obtain a test result; and

Step S14: automatically analyze the test result to provide a personalized test suggestion for the person to be tested.

In an embodiment, the step S10 can sense at least three feature points of the face of the person to be tested and compare these feature points with a plurality of feature point and eye position corresponding information stored in the database to find out the eye position information corresponding to the person to be tested, so that the positioning of the eyes of the person to be tested can be completed. These feature points can be selected from a forehead, a chin, a left ear, a right ear, a left cheek (a left cheekbone), a right cheek (a right cheekbone), a nose tip, a nose bridge, a left mouth corner, a right mouth corner, an upper lip and a lower lip, but not limited to this.

In another embodiment, the step S10 can also determine whether the person to be tested has used the automatic eye test device before according to the identity information (such as name, date of birth, fingerprint, voiceprint, retina, identity number, etc.) provided by the person to be tested. If the determination result is yes, the method can find out the eye position information corresponding to the person to be tested from the database to complete the positioning of the eyes of the person to be tested; if the determination result is no, the method will compare at least one of the identification items (such as gender, age, race, etc.) of the identity information with a plurality of identification item and eye position corresponding information stored in the database to find out the eye position information corresponding to the person to be tested to complete the positioning of the eyes of the person to be tested.

In practical applications, the step S10 can be operated through, for example, a six-degree-of-freedom (6-DOF) motion platform, but not limited to this; the step S12 can perform different tests on the eyes of the person to be tested according to actual needs, such as vision test, optometry, fundus photography, retinal optical coherence tomography (OCT), intraocular pressure test, corneal thickness test and/or corneal curvature test, but not limited to this; the personalized testing suggestions provided in the step S14 can include a suggestion for a testing appointment date and/or a suggestion for testing items, but not limited to this. When the test result obtained in the step S12 is determined to be unreliable, the step S12 can be automatically executed again.

Compared to the prior art, the automatic eye test device and method of the invention can automatically identify the identity of the person to be tested to obtain his/her previous eye positioning information to automatically complete the positioning of the eye of the person to be tested and automatically perform different tests on the eye of the person to be tested to obtain the test result, and they can also automatically analyze the personalized test suggestion (such as a suggestion for a testing appointment date and/or a suggestion for testing items) for the person to be tested based on the test result, so the person to be tested can use a single automatic eye test device to complete the entire eye test procedure by himself/herself. There is no need for professional testers to test the person to be tested through multiple eye test instruments, so it can greatly reduce the cost of manpower and equipment and effectively improve the popularity and convenience of eye test.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An automatic eye test device, comprising:
an automatic positioning module, configured to automatically complete a positioning of an eye of a person to be tested;
an automatic test module, coupled to the positioning module and configured to automatically test the eye of the person to be tested to obtain a test result; and
an automatic analysis module, coupled to the automatic test module and configured to automatically analyze the test result to provide a personalized test suggestion for the person to be tested.

2. The automatic eye test device of claim 1, wherein the automatic positioning module senses at least two feature points of a face of the person to be tested and compares the at least two feature points with a plurality of feature point and eye corresponding position information stored in a database to complete the positioning of the eye of the person to be tested.

3. The automatic eye test device of claim 1, wherein the automatic positioning module determines whether the person to be tested has used the automatic eye test device according to an identification information provided by the person to be tested; if a determination result of the automatic positioning module is yes, the automatic positioning module finds out an eye positioning information corresponding to the person to be tested from a database to complete the positioning of the eye of the person to be tested; if the determination result is no, the automatic positioning module compares at least one identification item in the identification information with a plurality of identification item and eye corresponding position information stored in the database to complete the positioning of the eye of the person to be tested.

4. The automatic eye test device of claim 2, wherein the at least two feature points are selected from a forehead, a chin, a left ear, a right ear, a left cheek, a right cheek, a left cheekbone, a right cheekbone, a nose tip, a nose bridge, a left mouth corner, a right mouth corner, an upper lip and a lower lip.

5. The automatic eye test device of claim 1, wherein the personalized test suggestion comprises a suggestion for a testing appointment date and/or a suggestion for testing items.

6. The automatic eye test device of claim 1, wherein the automatic test module performs vision test, optometry, fundus photography, retinal optical coherence tomography (OCT), intraocular pressure test, corneal thickness test and/or corneal curvature test on the eye of the person to be tested.

7. The automatic eye test device of claim 1, wherein when the test result is determined to be unreliable, the automatic test module automatically retests the eye of the person to be tested.

8. The automatic eye test device of claim 1, wherein the automatic positioning module is disposed on a six-degree-of-freedom (6-DOF) motion platform to operate.

9. An automatic eye test method, comprising steps of:
(a) automatically completing a positioning of an eye of a person to be tested;
(b) automatically test the eye of the person to be tested to obtain a test result; and
(c) automatically analyze the test result to provide a personalized test suggestion for the person to be tested.

10. The automatic eye test method of claim 9, wherein the step (a) comprises:
sensing at least two feature points of a face of the person to be tested and comparing the at least two feature points with a plurality of feature point and eye corresponding position information stored in a database to complete the positioning of the eye of the person to be tested.

11. The automatic eye test method of claim 9, wherein the step (a) comprises:
determining whether the person to be tested has used the automatic eye test device according to an identification information provided by the person to be tested; if a determination result of the step (a) is yes, finding out an eye positioning information corresponding to the person to be tested from a database to complete the positioning of the eye of the person to be tested; if the determination result is no, comparing at least one identification item in the identification information with a plurality of identification item and eye corresponding position information stored in the database to complete the positioning of the eye of the person to be tested.

12. The automatic eye test method of claim 9, wherein the at least two feature points are selected from a forehead, a chin, a left ear, a right ear, a left cheek, a right cheek, a left cheekbone, a right cheekbone, a nose tip, a nose bridge, a left mouth corner, a right mouth corner, an upper lip and a lower lip.

13. The automatic eye test method of claim 9, wherein the personalized test suggestion comprises a suggestion for a testing appointment date and/or a suggestion for testing items.

14. The automatic eye test method of claim 9, wherein the step (b) comprises:
performing vision test, optometry, fundus photography, retinal optical coherence tomography (OCT), intraocular pressure test, corneal thickness test and/or corneal curvature test on the eye of the person to be tested.
